(19) United States
(12) Patent Application Publication
(10) Pub. No.: US 2004/0126344 A1
(43) Pub. Date: Jul. 1, 2004

(54) Compositions having glycolipid to lighten skin and alter post-inflammatory hyperpigmentation

(75) Inventors: Harish Mahalingam, Ledgewood, NJ (US); Brian Jones, Warwick, NY (US); Gopinathan K. Menon, Wayne, NJ (US)

Correspondence Address: Charles N.J. Ruggiero, Esq., Ohlandt, Greeley, Ruggiero & Perle, L.L.P., One Landmark Square, 10th Floor, Stamford, CT, 06901-2682, US

(73) Assignee: Avon Products, Inc.

(21) Appl. No.: 10/330,039

(22) Filed: Dec. 26, 2002

(51) Int. Cl. A61K 7/135

(52) U.S. Cl. 424/62

(57) ABSTRACT

There is provided a composition for lightening skin having an effective amount of a glycolipid. There is also provided a composition for lightening skin having an effective amount of a glycolipid and a second lightening material. There is also provided methods for lightening skin including applying the compositions of the present invention to the skin.

### BACKGROUND OF THE INVENTION

1. Field of the Invention

The present invention relates to compositions and methods for lightening of the skin or reducing/altering post-inflammatory hyperpigmentation. More particularly, the present invention relates to compositions having a glycolipid, and more preferably a tomato glycolipid, and methods of application of same, for lightening the skin or reducing/altering post-inflammatory hyperpigmentation. Even more particularly, the tomato glycolipid is combined with another skin lightening ingredient to lighten skin and reduce/alter post-inflammatory hyperpigmentation.

2. Description of the Related Art

Whitening agents have been used in the past to lighten the skin for a variety of reasons. Dermatological conditions that are frequently treated with such agents include melasma, cholasma, freckles, pigmented keratoses, sun-induced damage, after-burn scars, post-injury hyperpigmentation, post-inflammatory hyperpigmentation, other hyperpigmentation, and age spots.

Skin pigmentation is determined by the level of melanin present in the epidermis. Three different types of melanin are present in, for example, the epidermis: DHI-melanin, DHICA-melanin and pheomelanin. The different types of melanin vary in color or shade. DHI-melanin is the darkest and is blackish in color. DHICA-melanin is brownish in color. Pheomelanin is the lightest and is reddish in color.

Melanin is synthesized in specialized organelles called melanosomes within pigment-producing cells (melanocytes). Melanocytes respond to stimuli to regulate melanin synthesis.

Topical lightening agents work to reduce pigmentation in varying ways. Some lightening agents act by interfering with the action of tyrosinase, the enzyme that catalyzes the conversion of the amino acid tyrosine to DOPAquinone. Others reduce pigmentation by inhibiting enzymes “downstream” from tyrosinase in the melanin synthesis pathway. These agents can regulate melanin synthesis by inhibiting DOPAchrome tautomerase and/or DHICA-polymerase. Other lightening agents work by inhibiting the uptake of melanosomes by keratinocytes.

It is desirable to have improved lightening agents that reduce pigmentation in skin.

Active ingredients derived from plants have commonly been employed in topical compositions for a myriad of medicinal, therapeutic and cosmetic purposes. Such actives can be obtained from various parts of a plant such as seeds, needles, leaves, roots, bark, cones, stems, rhizomes, callus cells, protoplasts, organs and organ systems, and meristems. Active ingredients are incorporated in such compositions in a variety of forms. Such forms include a pure or semi-pure component, a solid or liquid extract or derivative, or a solid plant matter. Plant matter may be incorporated in a variety of subforms such as whole, minced, ground or crushed.

Plant glycoproteins have been shown to inhibit melanosome transfer to keratinocytes in the skin. Minwalla et al., “Inhibition of Melanosome Transfer from Melanocytes to Keratinocytes by Lectins and Neoglycoproteins in an in Vitro Model System,” Pigment Cell Res, 14(3), 185-94 (June 2001).

U.S. Pat. No. 4,745,186 to Mudd, et al. describes methods for extracting glycolipids of a specific tomato plant, Lycopersicon pennellii Corr., and various topical compositions having the extract or a synthesized compound equivalent to the extract. However, tomato glycolipids have not been used for lightening skin.

Therefore, it would be desirable to have compositions that employ a tomato glycolipid that provides effective levels of lightening, bleaching, hypopigmenting, whitening and/or depigmenting (hereinafter referred to individually and collectively as “lightening” or “lighten”).

### SUMMARY OF THE INVENTION

It is an object of the present invention to provide compositions for lightening of the skin having an effective amount of a glycolipid.

It is another object of the present invention to provide compositions for lightening of the skin having an effective amount of a tomato glycolipid and an effective amount of a second lightening material.

It is further object of the present invention to provide methods for applying to the skin a composition of the present invention for lightening of the skin.

These and other objects and advantages of the present invention are provided by a composition for lightening skin having an effective amount of a glycolipid, preferably a tomato glycolipid. There is also provided a composition for lightening skin having an effective amount of a glycolipid and another lightening material. There are also methods for lightening skin using compositions having an effective amount of a glycolipid, preferably a tomato glycolipid.

### DETAILED DESCRIPTION OF THE INVENTION

The present invention is directed to compositions for lightening skin having an effective amount of a glycolipid. In a preferred embodiment, compositions for lightening skin of the present invention have an effective amount of a glycolipid and at least one other, or a second, lightening material. An effective amount means an amount sufficient to determine a perceptible change to the skin between prior to and after the application.

The glycolipids of the present invention are extracted glycolipids, preferably from plant origin. More preferably, the glycolipids are extracts of tomato leaves, thus called a tomato glycolipid.

Glycolipids alter the cell-cell recognition of endogenous sugars or natural glycolipids expressed on the surfaces of skin cells. Cell-cell recognition plays a role in transfer and uptake of melanosomes by keratinocytes. The rate of uptake, processing, and retention of melanosomes are responsible for visual manifestation of skin color. The glycolipids expressed by plants can be used as decoys to interfere with the cell-cell recognition process, thus decreasing the rate of uptake, processing, and retention of melanosomes producing a lightening of the skin.

A similar cell-cell recognition process involving cell surface glyco-moities also plays a role in irritant/immune response (Langerhan cell-keratinocyte interaction). The glycolipids of the present compositions may also interfere with this process and provide an anti-irritant/anti-inflammation benefit.

The glycolipids of the present invention are suitable for lightening skin. Most preferably, these glycolipids are extracted from tomato plants. An example of a glycolipid suitable for use in the present compositions, and a related method of extraction, are described in U.S. Pat. No. 4,745,186 to Mudd, et al. at column 4, line 66 to column 5, line 41. These glycolipids are 2,3,4-tri-O-acylhexoses extracted and purified from the epicuticular exudates of Lycopersicon pennellii plant parts.

In a most preferred embodiment, the present composition for lightening skin has an effective amount of a glycolipid and an effective amount of at least one second lightening material. In this embodiment, the glycolipid is a tomato glycolipid. This is the most preferred embodiment.

The second skin lightening material can be any material suitable for lightening skin and combinable with the glycolipids of the present invention. Some examples of such suitable second lightening materials include, but are not limited to, ascorbyl glucosides, vitamin C, retinol and retinol derivatives, water soluble licorice extracts, bearberry extracts, Rumex crispus extracts, milk proteins including hydrolyzed milk proteins, N,N,S-tris(carboxymethyl)cysteamines, oleanolic acids, perilla oils, placenta extract, Saxifragia sarmentosa, grape extract, Azadirachta indica A. Juss. Var., Glycyrrhiza glabra Linn., Morinda citrifolia Linn., Naringi crenulata (Roxb) Nicolson, Ligusticum chiangxiong Hort., Asmunda japonica Thumb., Stellaria medica (L.) cyr., Sedum sarmentosum Bunge, Ligusticum lucidum Ait., Ilex purpurea Hassk, Emblica, apigenin, ascorbyl palmitol, carruba polyphenol, hesperitin, hydroquinone, inabata polyphenol, isoliquirtigenin, kaempherol-7-neohesperidose, L-mimosine, luteolin, oil-soluble licorice extract, oxa acid, phenyl isothiocyanate, silymarin, T4CA, tetrahydro curcumin, unitrienol, ursolic-oleanolic acid, UVA/URSI, N,N,S-tris(carboxymethyl)cysteamine, cococin, TDPA, carboxycysteamine, cococin, perilla seed extract, perilla extract, juniperic acid, stenolama chusana (L.) ching, or any combinations thereof.

In any embodiment of the present compositions, the one or more glycolipids are present in an amount about 0.001 percentage by weight (wt %) to about 20 wt % based on the total weight of the composition. The one or more glycolipids are present preferably in an amount about 0.05 wt % to about 10 wt %, and more preferably in an amount about 0.1 wt % to about 5 wt %, based on the total weight of the composition.

In the more preferred compositions of the present invention in which the compositions also include one or more second lightening materials, the one or more second skin lightening materials are present in an amount about 0.001 wt % to about 20 wt % based on the total weight of the composition. The one or more second lightening materials are present preferably in an amount about 0.05 wt % to about 10 wt %, and more preferably in an amount about 0.1 wt % to about 5 wt %, based on the total weight of the composition.

The compositions of the present invention may have a carrier or vehicle. Preferably, the compositions of the present invention have a carrier or vehicle suitable for topical application. Examples of a carrier or vehicle suitable for use in the present compositions include, but are not limited to, water; vegetable oils; esters such as octyl palmitate, isopropyl myristate and isopropyl palmitate; ethers such as dicapryl ether and dimethyl isosorbide; alcohols such as ethanol and isopropanol; fatty alcohols such as cetyl alcohol, stearyl alcohol and behenyl alcohol; isoparaffins such as isooctane, isododecane and isohexadecane; silicone oils such as dimethicones and polysiloxanes; hydrocarbon oils such as mineral oil, petrolatum, isoeicosane and polyisobutene; polyols such as propylene glycol, glycerin, butylene glycol, pentylene glycol and hexylene glycol; or any combinations thereof.

The compositions of the present invention can be formulated in any suitable product form. Such product forms include, but are not limited to, aerosol spray, cream, dispersion, emulsion, foam, gel, liquid, lotion, mousse, ointment, pack, patch, pomade, powder, pump spray, solid, solution, stick, and towelette.

The present topical compositions may include one or more of the following: anesthetics, anti-allergenics, antifungals, antimicrobials, anti-inflammatory agents, antioxidants, antiseptics, chelating agents, colorants, emollients, emulsifiers, exfollients, film formers, fragrances, humectants, insect repellents, lubricants, moisturizers, pharmaceutical agents, photostabilizing agents, preservatives, skin protectants, skin penetration enhancers, sunscreens, stabilizers, surfactants, thickeners, viscosity modifiers, vitamins, or any combinations thereof.

The present invention also includes methods of lightening skin or reducing post-inflammatory hyperpigmentation. These methods involve the topical application of a composition having an effective amount of a glycolipid, preferably a tomato glycolipid.

The present invention further includes a method of lightening skin involving the topical application for an effective period of time of a composition having an effective amount of a glycolipid and an effective amount of a second lightening material. The present invention further includes a method of reducing post-inflammatory hyperpigmentation by the topical application for an effective period of time of a composition having an effective amount of a glycolipid and, more preferably, an effective amount of a second lightening material.

An effective period of time means that the composition is applied to the skin at least once daily for at least one week, preferably for at least two weeks, and more preferably for at least four weeks, to produce a visually perceptible lightening of the skin.

### EXAMPLE

Melanosome Uptake Assay (Tomato glycolipid) was done as follows. Confluent cultures of B16 melanocytes produce moderate levels of melanosomes. However, to induce elevated melanosome production in this cell line, semi-confluent (60%) cultures of B16 cells were treated for approximately thirty-six (36) hours with normal growth medium supplemented with 10 mM ammonium chloride (final conc.). The medium was then aspirated and the hypermelanotic cells were washed (2×2 ml) with distilled water to provide a hypotonic stress to the cells. An aliquot (2 ml) of a hypotonic lysis solution (0.02% NP-40 in water) was added to each plate and the plates were incubated for approximately five (5) minutes at room temperature. Following verification of cell lysis using light microscopy, the cellular material from three (3) culture plates were pooled in a 15 ml conical tube and centrifuged at approximately (200×g) for five (5) minutes to remove cellular debris. The resulting supernatant containing melanosomes was transferred to a clean 15 ml conical tube and centrifuged (850×g) for 20 minutes. The resulting pellet containing the isolated melanosomes were resuspended in 1 ml of Phosphate Buffered Saline (PBS) and stored at 4° C. until used.

The treatment of keratinocytes with melanosomes was measured as follows. The normal human epidermal keratinocytes (NHEKs) (available from Clonetics, Inc.) were plated in the wells of 24-well plates at a density of 200,000 cells/well. Approximately twenty-four (24) hours later, the growth medium was replaced with 1 ml of the appropriate growth medium (i.e., DMEM/KGM-2) containing the melanosome preparation with or without additional treatment conditions. The cells were treated with different concentrations of tomato glycolipid. The cells were then returned to the incubator for approximately one and one-half (1.5) hours. For these studies, each well of keratinocytes was treated with the amount of melanosomes isolated from a single plate of B16 cells.

In some experiments, the twenty-four (24) well plates of treated keratinocytes were centrifuged for 15 minutes at 1,000 rpm to facilitate the deposition of the melanosomes onto the surface membranes of the keratinocytes. The plates were then returned to the incubator for 1.25 hours.

For the analysis of melanosome uptake, the cells in each well of keratinocytes were rinsed (3×1 ml) with PBS, removed from the plate using trypsin/EDTA, washed with PBS. To analyze the uptake of melanosomes by the keratinocytes, the internalized melanin was extracted from the cells according to a modified method of Bessou-Touya, S., et al. (Chimeric human epidermal reconstructs to study the role of melanocytes and keratinocytes in pigmentation and photoprotection. J. Invest. Dermatol., 111:1103-1108, 1998) and quantified spectrophotometrically by determining the melanin-specific absorbance at 405 nm.

Melanocytes synthesize melanin and deposits onto melanosomes. Visual manifestation of skin color is due to presence of melanin/melanosomes in keratinocytes. Melanosomes are taken up by keratinocytes and the rate of uptake, retention and processing of melanosomes in the keratinocytes is a key determinant of skin color. The internalized melanin value reflects the amount of melanin/melanosome uptake and retention by the keratinocytes. Thus, lower internalized melanin values, particularly internalized melanin values that are less than the control with melanin, indicate that melanin uptake by the keratinocytes has been inhibited.

The results are as follows. At 0.5% volume/volume, tomato glycolipid showed a marked visual decrease in melanosome uptake as compared to the positive control.

It should be understood that the foregoing description is only illustrative of the present invention. Various alternatives and modifications can be devised by those skilled in the art without departing from the invention. Accordingly, the present invention is intended to embrace all such alternatives, modifications and variances which fall within the scope of the appended claims.

What is claimed is:

1. A topical composition for lightening skin comprising:
a glycolipid in an amount effective to lighten skin; and
a cosmetically acceptable vehicle.

2. The composition of claim 1, wherein the glycolipid is a tomato glycolipid.

3. The composition of claim 1, wherein the glycolipid is present in an amount about 0.001 wt % to about 20 wt % based on the total weight of the composition.

4. The composition of claim 1, wherein the glycolipid is present in an amount about 0.05 wt % to about 10 wt % based on the total weight of the composition.

5. The composition of claim 1, wherein the glycolipid is present in an amount about 0.1 wt % to about 5 wt % based on the total weight of the composition.

6. A topical composition for lightening skin comprising:
a glycolipid; and
a second lightening material,
wherein the glycolipid and the second lightening material are present in amounts effective to lighten the skin after the composition is applied to the skin.

7. The composition of claim 6, wherein the glycolipid is a tomato glycolipid.

8. The composition of claim 6, wherein the glycolipid is present in an amount about 0.001 wt % to about 20 wt % based on the total weight of the composition.

9. The composition of claim 6, wherein the glycolipid is present in an amount about 0.05 wt % to about 10 wt % based on the total weight of the composition.

10. The composition of claim 6, wherein the glycolipid is present in an amount about 0.1 wt % to about 5 wt % based on the total weight of the composition.

11. The composition of claim 6, wherein the second lightening material is present in an amount about 0.001 wt % to about 20 wt % based on the total weight of the composition.

12. The composition of claim 6, wherein the second lightening material is present in an amount about 0.05 wt % to about 10 wt % based on the total weight of the composition.

13. The composition of claim 6, wherein the second lightening material is selected from the group consisting of ascorbyl glucosides, vitamin C, retinol and retinol derivatives, water soluble licorice extracts, bearberry extracts, Rumex crispus extracts, milk proteins including hydrolyzed milk proteins, N,N,S-tris(carboxymethyl)cysteamines, oleanolic acids, perilla oils, placenta extract, Saxifragia sarmentosa, grape extract, Azadirachta indica A. Juss. Var., Glycyrrhiza glabra Linn., Morinda citrifolia Linn., Naringi crenulata (Roxb) Nicolson, Ligusticum chiangxiong Hort., Asmunda japonica Thumb., Stellaria medica (L.) cyr., Sedum sarmentosum Bunge, Ligusticum lucidum Ait., Ilex purpurea Hassk, Emblica, apigenin, ascorbyl palmitol, carruba polyphenol, hesperitin, hydroquinone, inabata polyphenol, isoliquirtigenin, kaempherol-7-neohesperidose, L-mimosine, luteolin, oil-soluble licorice extract, oxa acid, phenyl isothiocyanate, silymarin, T4CA, tetrahydro curcumin, unitrienol, ursolic-oleanolic acid, UVA/URSI, N,N,S-tris(carboxymethyl)cysteamine, cococin, TDPA, carboxycysteamine, cococin, perilla seed extract, perilla extract, juniperic acid, stenolama chusana (L.) ching, or any combinations thereof.

14. The composition of claim 6, further comprising a cosmetically acceptable vehicle.

15. A method of lightening skin comprising topically applying to the skin for an effective period of time an effective amount of a composition having a glycolipid and a cosmetically acceptable vehicle.

16. The method of claim 15, wherein the composition has a second lightening material, and wherein the glycolipid and the second lightening material are present in amounts effective to lighten the skin after the composition is applied to the skin.

17. The method of claim 16, wherein the glycolipid is a tomato glycolipid.

18. The method of claim 17, wherein the glycolipid is present in an amount about 0.001 wt % to about 20 wt % based on the total weight of the composition.

19. The method of claim 18, wherein the second lightening material is present in an amount about 0.001 wt % to about 20 wt % based on the total weight of the composition.

20. The method of claim 17, wherein the glycolipid is present in an amount about 0.05 wt % to about 10 wt % based on the total weight of the composition.

21. The method of claim 20, wherein the second lightening material is present in an amount about 0.05 wt % to about 10 wt % based on the total weight of the composition.

22. The method of claim 17, wherein the glycolipid is present in an amount about 0.1 wt % to about 5 wt % based on the total weight of the composition.

23. The method of claim 22, wherein the second lightening material is present in an amount about 0.1 wt % to about 5 wt % based on the total weight of the composition.

24. The method of claim 15, wherein the composition is applied at least once daily for a period of at least one week.

25. The method of claim 15, wherein the composition is applied at least once daily for a period of at least two weeks.

26. The method of claim 15, wherein the composition is applied at least once daily for a period of at least four weeks.

27. A method of reducing post-inflammatory hyperpigmentation comprising topically applying to the skin for an effective period of time an effective amount of the composition of claim 6.

28. The method of claim 27, wherein the composition is applied at least once daily for a period of at least one week.

29. The method of claim 27, wherein the composition is applied at least once daily for a period of at least two weeks.

30. The method of claim 27, wherein the composition is applied at least once daily for a period of at least four weeks.
